(19) United States
(12) Patent Application Publication
(10) Pub. No.: US 2017/0174530 A1
(43) Pub. Date: Jun. 22, 2017

(54) Oil-In-Water Monitoring

(71) Applicants: Xiaolei Yin, Conroe, TX (US); Michael D. Olson, Fort Worth, TX (US); Edward J. Grave, Montgomery, TX (US); Kamran Ahmed Gul, The Woodlands, TX (US)

(72) Inventors: Xiaolei Yin, Conroe, TX (US); Michael D. Olson, Fort Worth, TX (US); Edward J. Grave, Montgomery, TX (US); Kamran Ahmed Gul, The Woodlands, TX (US)

(21) Appl. No.: 15/367,040

(22) Filed: Dec. 1, 2016

Related U.S. Application Data
(60) Provisional application No. 62/268,590, filed on Dec. 17, 2015.
(60) Provisional application No. 62/424,137, filed on Nov. 18, 2016.

(51) Int. Cl. C02F 1/00; B01D 17/04; G01N 33/18; E21B 43/40; E21B 49/08; E21B 43/36; B01D 17/12; C02F 1/40

(52) U.S. Cl. CPC C02F 1/008; B01D 17/12; B01D 17/045; C02F 1/40; E21B 43/40; E21B 49/08; E21B 43/36; G01N 33/1833; G01N 33/1893; C02F 2209/42; C02F 2303/16; C02F 2209/40; C02F 2103/10

(57) ABSTRACT

An oil-in-water monitoring (OIWM) system for monitoring an oil-in-water concentration of treated water in a subsea processing system. The system includes a first OIWM portion including a separation component configured to separate the treated water stream into a separated oil portion and a separated water portion which has a plenum including the separated oil portion and the separated water portion. A separation component instrument is operatively coupled to the plenum. The system also includes at least two of: an oil line instrument operatively coupled to an oil line, a water line instrument operatively coupled to a water line, and an inlet line instrument operatively coupled to the inlet line. A computational device is configured to output an oil-in-water concentration of the inlet treated water stream using the parameters measured by the separation component instrument and at least two of the other instruments. Methods using such systems are also disclosed.

### CROSS-REFERENCE TO RELATED APPLICATION

This application claims the benefit of U.S. Provisional Patent Application Ser. No. 62/268,590, filed Dec. 17, 2015, entitled “Oil-In-Water Monitoring and Online Calibration of Oil-In-Water Monitors,” and U.S. Provisional Patent Application Ser. No. 62/424,137, filed Nov. 18, 2016, entitled “Oil-In-Water Monitoring,” the entireties of which are incorporated by reference herein.

### BACKGROUND

Recent developments and advances in exploration, drilling, and processing technologies have enabled operating companies in the oil and gas industry to maintain hydrocarbon production in maturing fields or bring new opportunities online. As the oil and gas industry moves to increasingly deeper water depths and/or longer tieback distances for hydrocarbon production, the technologies that enable economically viable development for subsea fields are becoming more attractive.

One particular technology, subsea processing, has gained significant interest from the oil and gas industry as an interesting field development option at least in part due to the following reasons: (i) subsea processing, including boosting, may increase production rates and recoverable reserves by reducing backpressure on subsea wells, (ii) water separation from the produced water streams may mitigate certain flow assurance issues, especially over long tieback distances, and (iii) subsea processing may enable fewer topsides facilities, smaller flowlines, and less energy requirements than multiphase boosting of the full well stream alone.

Subsea separation technologies endeavor to separate produced water from the full well stream at the sea bed. For subsea processing systems with long tieback distances or deeper water applications, reinjection of produced water may be a cost-effective way of disposing produced water after separation. Additionally, reinjection of produced water may enable improved hydrocarbons recovery by eliminating the need for transporting non-sale product (e.g., water) to topsides or onshore facilities over long tie-back distances.

However, the produced water leaving the subsea separators may be at least partially a multiphase fluid containing some level of dispersed oil. Dispersed oil in produced water may be substantially removed before reinjection because it can decline well injectivity. Specifically, even small amounts of dispersed oil, when injected with produced water, can increase oil saturation in the near-wellbore region and decrease the effective permeability of formation to injection water. Over time, this decrease in permeability may cause a partial loss in well injectivity and even, in some cases, a complete loss of the injection well. To mitigate this risk, and depending on the reservoir porosity and permeability, common practices may reduce the oil-in-water (OIW) concentration allowed in the reinjection water to less than 400 parts per million (ppm) in volume. To achieve this level, water treatment systems are commonly used. Water treatment systems may include a single- or multi-stage de-oiling systems that reduce oil content in produced water to meet the reinjection water quality requirements. Sometimes de-sanding systems are added to remove the solids content that may be present in produced water.

To ensure that the quality of the injection water meets reinjection requirements for a particular formation, subsea oil-in-water monitoring can be used to measure the oil content in produced water. The accurate measurement of oil content in the produced water presents various technical challenges. For example, oil-in-water monitoring technologies used in topside or on-shore facilities require frequent cleaning and calibration to ensure reading accuracy. However, subsea processing equipment may not be easily accessible, leading to errors in measurements (e.g., due to clogging, fouling, etc.), errors in calibration (e.g., with accompanying sampling), etc. Consequently, subsea sampling technologies and techniques are likely to be relatively more costly and less accurate compared to topsides or on-shore sampling programs.

Conventional methods for calibrating subsea sensors have been devised and include: (i) use of sample lines from subsea to topsides to provide water samples for reference measurements, and (ii) use of a subsea sampling systems to collect water samples. Use of sample lines may cause delays in measurement because water samples are required to travel along the sample lines. This may pose flow assurance risks such as clogging, plugging, or other flow inhibition of the sampling lines, and may carry significant equipment line costs for long tie-back distances. In addition, the oil composition may change (e.g., oil aging) from subsea to topsides which may introduce additional errors in reference measurements. Alternatively, use of subsea sampling systems to collect water samples may present different challenges. For example, some subsea sampling systems may only provide one-time sampling. This may be insufficient for suitable sensor calibration. Additionally, subsea sampling systems may be costly, may have a large footprint, and/or may require a remotely operated vehicle (ROV) to carry the samples to topsides. These and other complications are well-known in the art and create a long-felt desire for improved sampling solutions to determine the concentration of oil within a water sample.

Therefore, a desire exists for a relatively less expensive, reliable subsea solution to overcome the disadvantages of the conventional approaches. Such a solution may desirably be an in-situ primary measurement method with a secondary measurement method which is relatively low cost compared to existing sampling methods for sensor calibration, and can provide additional measurements for comparison with the primary measurement method. Such a solution may desirably include the ability to perform online calibration of existing oil-in-water monitors. Such a solution may desirably include a distinct oil-in-water monitoring approach to avoid possible errors in methodology. Such a solution may desirably provide relatively fast analysis as compared to conventional approaches.

### SUMMARY OF THE INVENTION

The present disclosure relates to an oil-in-water monitoring (OIWM) system. In one aspect, the present disclosure relates to an OIWM system including a first OIWM portion. The first OIWM portion includes a separation component in fluid communication with an inlet line and configured to separate the treated water stream into a separated oil portion and a separated water portion. The separation component includes a plenum including the separated oil portion and the separated water portion. The first OIWM portion further includes a separation component instrument operatively coupled to the plenum and configured to measure a parameter associated with the separated oil portion, the separated water portion, or both within the plenum.

The system also includes an oil line in fluid communication with the separation component and configured to pass a first stream comprising the separated oil out of the first OIWM portion and a water line in fluid communication with the separation component and configured to pass a second stream comprising the separated water portion out of the first OIWM portion. The system also includes at least two of: an oil line instrument operatively coupled to the oil line and configured to measure a parameter associated with the separated oil portion within the oil line, a water line instrument operatively coupled to the water line and configured to measure a parameter associated with the separated water portion within the water line, and an inlet line instrument operatively coupled to the inlet line and configured to measure a parameter associated with the treated water stream within the inlet line.

The system also includes a computational device operatively coupled to the separation component instrument and at least two of: the oil line instrument, the water line instrument, and the inlet line instrument and configured to output an oil-in-water concentration of the treated water stream using the parameters measured by the separation component instrument and at least two of the other instruments.

The OIWM system may additionally include a second OIWM portion. The second OIWM portion may include an OIWM sensor configured to receive the treated water stream. The second OIWM portion may include an OIWM sensor. The second OIWM portion may be configured to determine an oil-in-water concentration of the treated water stream. Optionally, the computational device or a control system including the computation device may be configured to compare the oil-in-water concentration of the first OIWM portion with the oil-in-water concentration of the second OIWM portion. The comparison may be used to calibrate the OIWM sensor of the second OIWM portion.

In another aspect, the present disclosure relates to a method of monitoring an oil-in-water concentration of treated water in a subsea processing system, the method comprising passing a first portion of the treated water to a first OIWM portion and separating the first portion of the treated water into a separated oil portion and a separated water portion using a separation component within the first OIWM portion. The method also includes measuring an interface level in the separation component and at least two additional parameters associated with the separated oil portion, the separated water portion, and the first portion of the treated water. The method produces a first result indicating an oil-in-water concentration of the first portion of the treated water using the interface level and the at least two additional parameters and compares the first result to a second result indicating an oil-in-water concentration of a second portion of the treated water, wherein the second result is obtained at a second OIWM portion including an OIWM sensor.

In yet another aspect, the present disclosure relates to an oil-in-water monitoring (OIWM) system, the system comprising a first OIWM portion configured to receive at least a first portion of a treated water stream passing from an outlet of a water treatment portion. The first OIWM portion is further configured to measure a plurality of parameters which are used to determine an oil-in-water concentration of the treated water stream, the first OIWM portion comprising a coalescer configured to receive the first portion of the treated water stream via an inlet line. The coalescer is configured to separate the first portion of the treated water stream into a separated oil portion and a separated water portion and includes a plenum which includes the separated oil portion and the separated water portion. The first OIWM portion also includes a coalescer instrument operatively coupled to the plenum and configured to measure an interface level within the plenum. The first OIWM portion has an oil line and a water line in fluid communication with the coalescer. The oil line is configured to pass a first stream comprising the separated oil portion out of the first OIWM portion and the water line is configured to pass a second stream comprising the separated water portion out of the first OIWM portion. The first OIWM portion also includes at least two of: a first flow meter, a second flow meter and a third flow meter. The first flow meter is operatively coupled to the oil line and is configured to measure a first flow rate associated with the separated oil portion within the oil line. The second flow meter is operatively coupled to the water line and is configured to measure a second flow rate associated with the separated water portion within the water line. The third flow meter is operatively coupled to the inlet line and is configured to measure a third flow rate associated with the first portion of the treated water stream within the inlet line. The OIWM system includes a computational device operatively coupled to the coalescer instrument and at least two of: the first flow meter, the second flow meter, and the third flow meter. The computational device is configured to calculate an oil-in-water concentration of the first portion of the treated water stream using the interface level and at least two of: the first flow rate, the second flow rate, and the third flow rate. The OIWM system includes a second OIWM portion configured to receive at least a second portion of the treated water stream passing from the outlet of the water treatment portion, wherein the second OIWM portion includes an OIWM sensor and is further configured to measure an oil-in-water concentration of the second portion of the treated water stream.

### DESCRIPTION OF THE DRAWINGS

To aid in the present disclosure, certain figures, illustrations, and/or flow charts are appended hereto. It is to be noted, however, that the drawings illustrate only selected embodiments of the inventions and are therefore not to be considered limiting of scope, for the inventions may admit to other equally effective embodiments and applications.

FIG. 1 is a simplified process flow diagram of a water treatment system.

FIG. 2 is a schematic diagram of an oil-in-water monitor (OIWM) system including two OIWM portions and a water treatment portion.

FIG. 3 is a schematic diagram of another embodiment of an OIWM system including two OIWM portions and a water treatment portion.

FIG. 4 is a block diagram depicting a method of monitoring an oil-in-water concentration of treated water in a subsea processing system.

FIG. 5 is a schematic diagram of an OIWM system including three OIWM portions.

FIG. 6 is a block diagram of an OIWM system including two OIWM portions.

FIG. 7 is a block diagram of an OIWM system including three OIWM portions.

FIG. 8 is a block diagram of an OIWM system including two OIWM portions.

FIG. 9 is a block diagram of an OIWM system including two OIWM portions.

### DETAILED DESCRIPTION

In the following detailed description section, specific embodiments of the present techniques are described. However, to the extent that the following description is specific to a particular embodiment or a particular use of the present techniques, this is intended to be for exemplary purposes only and simply provides a description of the exemplary embodiments. Accordingly, the techniques are not limited to the specific embodiments described herein, but rather, include all alternatives, modifications, and equivalents falling within the true spirit and scope of the appended claims.

At the outset, for ease of reference, certain terms used in this application and their meanings as used in this context are set forth. To the extent a term used herein is not defined herein, it should be given the broadest definition persons in the pertinent art have given that term as reflected in at least one printed publication or issued patent. Further, the present techniques are not limited by the usage of the terms shown herein, as all equivalents, synonyms, new developments, and terms or techniques that serve the same or a similar purpose are considered to be within the scope of the present disclosure.

As used herein, the terms “a” and “an”, mean one or more when applied to any feature in embodiments of the present inventions described in the specification and claims. The use of “a” and “an” does not limit the meaning to a single feature unless such a limit is specifically stated.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one”, in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C,” “one or more of A, B, or C” and “A, B, and/or C” may mean A alone, B alone, C alone, A and B together, A and C together, B and C together, A, B and C together, and optionally any of the above in combination with at least one other entity.

As used herein, the term “fluid” may refer to gases, liquids, and combinations of gases and liquids, as well as to references to the same with or without solid particulate (e.g., sand).

As used herein, the term “hydrocarbon” means an organic compound that includes primarily, if not exclusively, the elements hydrogen and carbon. Hydrocarbons generally fall into two classes: aliphatic, or straight chain hydrocarbons, and cyclic, or closed ring, hydrocarbons including cyclic terpenes. Examples of hydrocarbon-containing materials include any form of natural gas, oil, and bitumen that can be used as a fuel or upgraded into a fuel.

As used herein, a “multiphase fluid” means a fluid that is amenable to flow and that is composed of two phases that are not chemically related (e.g., oil and water) or where more than two phases are present (e.g., liquid and gas), depending on context, irrespective of whether the multiphase fluid comprises trace amounts of a particular phase or substantial amounts of the particular phase.

As used herein, “substantially”, “predominately” and other words of degree are relative modifiers intended to indicate permissible variation from the characteristic so modified. It is not intended to be limited to the absolute value or characteristic which it modifies, but rather possessing more of the physical or functional characteristic than its opposite, and preferably, approaching or approximating such a physical or functional characteristic.

As used herein, the definite article “the” preceding singular or plural nouns or noun phrases denotes a particular specified feature or particular specified features and may have a singular or plural connotation depending upon the context in which it is used.

The present disclosure includes techniques for using an online, in-situ separation component, e.g., a coalescer, a filter, etc., to separate a portion of a treated water stream into a separated oil phase portion and a separated water phase portion. The resulting level of the separated oil and water within the separation component is measured and used to determine volumes of the oil phase and the water phase remaining in the separation component. The volumes of the oil phase and the water phase that exit the separation component are determined by the additional measured parameters, e.g., flow rates, in at least two of the oil line, the water line, and the inlet line. Based on all the volumes of the separated oil and the separated water, an oil-in-water concentration of the treated water stream entering the separation component can be determined. The oil-in-water concentration may be compared to the oil-in-water concentration of another portion of the treated water stream determined by a primary oil-in-water monitoring (“OIWM”) portion for error detection, calibration, maintenance, etc. Alternately and/or additionally, the disclosed approach may be utilized as a backup to a primary OIWM portion. The disclosed techniques may afford a relatively less expensive, reliable subsea solution to overcome the disadvantages of the conventional approaches. The disclosed techniques may provide an in-situ reference measurement method with relatively low cost, and may provide the capability for continuous sampling. The disclosed techniques may provide the ability to perform online calibration of primary oil-in-water monitors used to determine the oil-in-water concentration of the treated water stream. The disclosed techniques may provide a distinct oil-in-water monitoring approach to avoid possible errors in methodology of conventional OIWM systems. The disclosed techniques may provide relatively fast analysis as compared to conventional approaches. The disclosed techniques may provide periodic and/or continuous sampling of a treated water volume. The disclosed techniques may include self-cleaning and/or back-flushing systems for providing increased reliability, accuracy, etc.

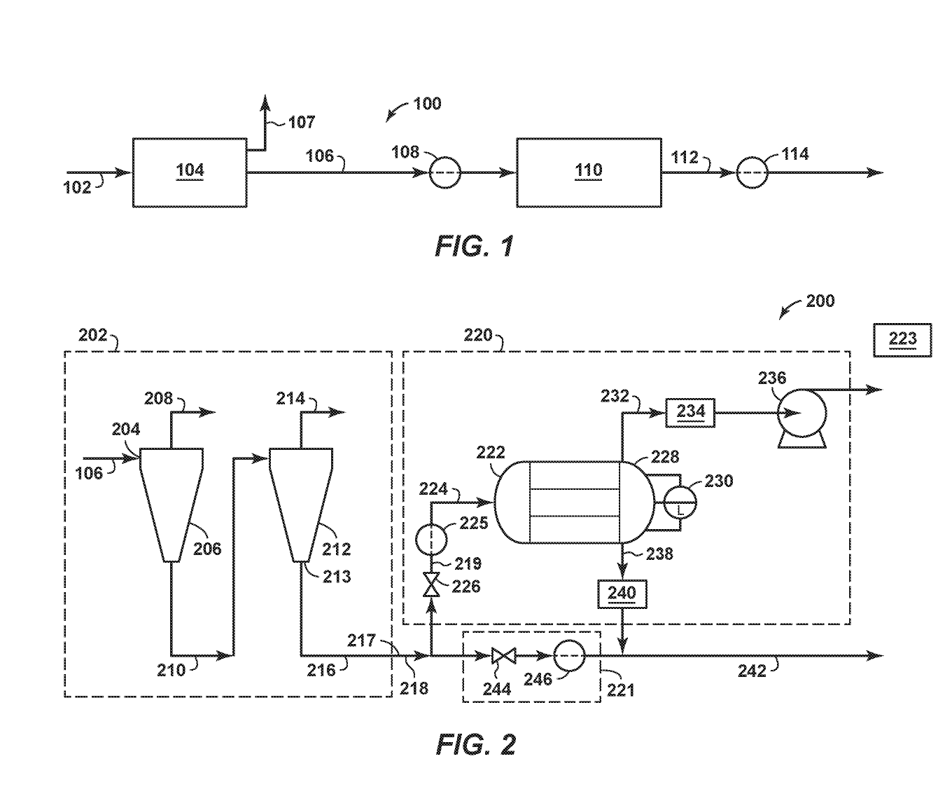

FIG. 1 is a simplified process flow diagram of a water treatment system 100. A separator 104 may receive a multiphase fluid stream 102. The multiphase fluid stream 102 received by the separator 104 may be any type of fluid that includes a water phase component and an oil phase component that are relatively immiscible. For example, the multiphase fluid may be production fluids from a subsea well. The multiphase production fluid stream 102 may comprise hydrocarbon fluids that include a mixture of natural gas, crude oil, brine, and/or solid impurities (such as sand), etc. The production fluid stream 102 may be obtained from a subsea well via any type of subsea production system (not depicted) that is configured to produce hydrocarbons from subsea locations. A gas-liquid separation system (not depicted) can optionally be used upstream to separate a gas phase component from the production fluid stream.

The main separator 104 may be an oil/water separator configured to achieve bulk separation of the multiphase production fluid stream 102 into a produced oil stream 107 and a produced water stream 106. Additional components, e.g., oil and water pre-treating or coalescence equipment, such as heating systems, chemical injection systems, electrostatic coalescing devices, cyclones for oil-water separation, and/or liquid export pipelines and the like may each be used in addition to these separation techniques.

The separator 104 may pass the produced water stream 106 through an oil-in-water monitor (OIWM) sensor 108 to a water treatment portion (section) 110. The OIWM sensor 108 may be used to determine the oil-in-water concentration of the produced water stream 106 prior to entering the water treatment portion 110. The water treatment portion 110 may include a single-stage, multi-stage, or other de-oiling system to reduce the oil content in the produced water stream 106 to provide a treated water stream 112 meeting the water quality requirements for reinjection, discharge, etc., e.g., between 0 and 400 parts per million (ppm), 200 ppm, 100 ppm, 50 ppm, 40 ppm, 30 ppm, 20 ppm, or lower. A de-sanding system (not shown) may be also added to remove the solids content that may be present in the produced water stream, the treated water stream, and/or the separated water portion.

The water treatment portion 110 may pass a treated water stream 112 to a second OIWM sensor 114 positioned downstream from the water treatment portion 110. The OIWM sensors may be selected from a fluorescence sensor, an acoustic sensor, an optical sensor, and any combinations thereof. A fluorescence sensor measures the amount of fluorescence emitted from oil contained within the treated water stream and determines the oil-in-water concentration by correlating the fluorescence emitted to an oil-in-water concentration. An acoustic sensor emits an acoustic signal into the treated water stream, measures an acoustic signal reflected from oil droplets in of the water phase, and determines the oil-in-water concentration by correlating the acoustic signals to an oil-in-water concentration. An optical sensor takes images of the oil droplets within the water phase and determines the oil-in-water concentration by analyzing the images to count and size the oil droplets and correlating the sized oil droplets to an oil-in-water concentration. The second OIWM sensor 114 may pass the output stream to a system outlet, e.g., combining at least a portion of the treated water stream 112 with at least one of a downstream reinjection water stream, discharge water stream, recirculation (recycle) stream, and combinations thereof. The second OIWM sensor 114 may be used to determine the oil-in-water concentration of the treated water stream 112 leaving the water treatment portion 110. A comparison of the first OIWM sensor 108 and the second OIWM sensor 114 may provide a measure of the efficacy or other operational characteristic of the water treatment portion 110 and/or the main separator 104. In some embodiments, the second OIWM sensor 114 may be useful in determining whether to optionally reinject, discharge, and/or recycle the treated water stream 112.

FIG. 2 is a schematic diagram of a system including an OIWM system 200. The system depicted in FIG. 2 optionally comprises a water treatment portion 202 configured to receive at least a portion of the produced water stream 106 from the main separator (not shown) at inlet 204 to a first stage de-oiling hydrocyclone 206. The first stage de-oiling hydrocyclone 206 separates the produced water stream into an oil portion and a water portion. The first stage de-oiling hydrocyclone 206 may pass a predominantly oil stream via reject line 208. The first stage de-oiling hydrocyclone 206 may pass a predominantly water stream via line 210 to a second stage de-oiling hydrocyclone 212. The second stage de-oiling hydrocyclone 212 receives the predominantly water stream via line 210 and separates the received predominantly water stream into an oil portion and a water portion. The second stage de-oiling hydrocyclone 212 may pass a predominantly oil stream via reject line 214. The second stage de-oiling hydrocyclone 212 may pass a treated water stream comprising predominantly water via an outlet 213 into outlet line 216.

An inlet line 218 may receive the treated water stream 217 from the water treatment portion 202 and may pass at least a portion (first portion) of the treated water stream 219 to a first OIWM portion (section) 220 of OIWM system 200 including a separation component which is depicted as a coalescer 222. Although the separation component is depicted in the figures as a coalescer, it is understood that any other separation component configured to separate the oil phase portion from the water phase portion of a treated water stream may be used. The treated water stream may comprise predominantly water with a measureable amount of oil. The first OIWM portion 220 may pass the received treated water stream 219 to coalescer 222 via an inlet line 224 comprising an isolation valve 226 which isolatably couples the inlet line 224 to the first OIWM portion 220 from the main inlet line 218. The coalescer 222 is in fluid communication with the inlet line 224 and main inlet line 218. As described further herein, the inlet line 224 may be a side-stream from the main inlet line 218. The coalescer 222 may be a high-efficiency multi-stage filter-based coalescer. Some embodiments may equip the coalescer 222 with coalescing and flow distribution internals, e.g., plate packs, perforated baffles, and the like, which may enhance coalescence and oil/water separation. Some embodiments may alternately or additionally utilize one or more other components within the first OIWM portion, such as membrane-based systems or additional coalescer systems, in series and/or parallel (e.g., to increase run length) to separate oil from water. All such alternate embodiments are considered within the scope of the present disclosure.

The coalescer 222 may be configured to separate and collect an amount of entrained oil. The coalescer 222 creates a separated oil portion and a separated water portion. The coalescer 222 comprises a plenum and/or chamber 228 which includes the separated oil portion and the separated water portion of the treated water stream 219 entering the coalescer 222. The separated oil portion may accumulate at the top part of the plenum 228, e.g., behind the filters, due to its lower density compared to the separated water portion.

A separation component instrument 230 is operatively coupled to the plenum 228 to measure a parameter associated with the separated oil portion, the separation water portion, or both within the plenum 228, e.g., the separation component instrument may be a level meter to measure the level of the oil-water interface in the plenum 228. Those of skill in the relevant art will appreciate that other instruments may be suitably employed within the first OIWM portion 220 to measure two or more parameters associated with the separated oil portion, the separated water portion, and the treated water stream entering the first OIWM 220 via inlet line 224. The separation component instrument 230 may be a level meter, a differential pressure sensor, a level profiler, or another suitable instrument for directly or indirectly providing oil-water interface measurements. The level measurement may be converted into the volume of oil content and water content accumulated in the plenum 228. The separation component instrument 230 may be configured to send a signal to a computational device 223. The computational device 223 is operatively coupled to the separation component instrument 230. Although the computational device 223 is depicted outside of the first OIWM portion and the second OIWM portion, it is understood the computational device 223 may be located at any suitable position within the system. The computational device includes a processor, memory, code within a non-transitory, computer-readable medium, and interface(s) configured to receive signal data and provide outputs. The output may include oil-in-water concentrations, data sets, command signals for the controller(s) within the OIWM system, or the like. The memory may be used to store data, code, and the like. The code is configured to direct the processor to execute commands, such as determining oil-in-water concentrations as discussed further herein. The computational device may be part of an overall control system or may be a separate component in communication with the overall control system. The computational device may be located subsea, topside, or any other suitable location. The control system may also include one or more controllers (not shown) associated with the various components of the OIWM system 200. The control system may also be operatively coupled to various other components to allow the controller take various actions, e.g., to adjust flow rates to obtain the desired operating characteristics, to generate alarms, to create records for long-term analysis, etc. The control system may be a distributed control system (DCS), a programmable logic controller (PLC), a direct digital controller (DDC), or any other appropriate control system. In some embodiments, the controller may automatically adjust parameters via controller outputs (not shown), or may provide information about the OIWM system 200 to an operator who then manually inputs adjustments.

The coalescer 222 may pass a first stream of the separated oil portion via an oil line 232 out of the first OIWM portion 220. Coalescer 222 is in fluid communication with the oil line 232. An oil line instrument 234 (e.g., a first flow meter) is operatively coupled to the oil line 232 and may measure a parameter, e.g., the volumetric flow rate, associated with the separated oil portion that exits the first OIWM portion. The oil line instrument 234 may be configured to send a signal to computational device 223 in substantially the same manner as the separation component instrument 230. A pump 236 may be placed along the oil line 232, e.g., to provide head to reintroduce the separated oil portion to the produced oil stream (not shown) or send to another location.

The coalescer 222 may pass a second stream of the separated water portion via a water line 238 out of the first OIWM portion 220. Coalescer 222 is in fluid communication with the water line 238. A water line instrument 240 (e.g., a second flow meter) is operatively coupled to the water line 238 and may measure a parameter, e.g., the volumetric flow rate, associated with the separated water portion that exits the first OIWM portion. The water line instrument 240 may be configured to send a signal to computational device 223 in substantially the same manner as the separation component instrument 230 and/or the oil line instrument 234.

Some embodiments may comprise additional or alternate instruments within the scope of this claims. For example, as shown in FIG. 2, inlet line instrument 225 (e.g., a third flow meter) is operatively coupled to the inlet line 224, installed upstream of the coalescer 222, and may measure a parameter associated with the treated water stream, e.g., the total volumetric flow of the treated water stream fed into the coalescer 222, potentially eliminating the need for a water line instrument 240 in the water line 238 for the separated water portion or an oil line instrument 234 in the oil line 232 for the separated oil portion.

The computational device is operatively coupled to and configured to receive signals from the separation component instrument 230 and at least two of the instruments 234, 240, 225 and utilizes the parameter signals from the separation component instrument 230 and at least two of the other instruments 234, 240, 225 to determine and output an oil-in-water concentration of the treated water stream 219. For example, the total volume of water passing through the coalescer 222 may be calculated as the sum of a water volume determined from a parameter measured by the instrument 240 and the water volume remaining in the plenum or chamber 228 determined from a parameter measured by the separation component instrument 230. The oil volume may be calculated as the sum of the oil volume determined from a parameter measured by the instrument 234 and the oil volume remaining in the plenum or chamber 228 determined from a parameter measured by the separation component instrument 230. The oil-in-water concentration may be calculated as the ratio of the oil volume to the total volume of oil and water. The computational device is configured to determine and output oil-in-water concentrations. The computational device may also be configured to determine and output an average of at least a portion of the determined oil-in-water concentrations for the portion of the OIWM system. The computational device may be further configured to compare an average of at least a portion of the determined oil-in-water concentrations for the first portion of the treated water stream with an average of at least a portion of the determined oil-in-water concentrations for the second portion of the treated water stream and determine and output the difference between the averages. Other calculation techniques will be apparent to those of skill in the relevant art, e.g., subtracting water volume from total volume to obtain oil volume, etc., and are considered within the scope of the present disclosure. These and other measurements may be optionally utilized for various purposes, e.g., as a reference to calibrate one or more OIWM sensors.

In some embodiments, the parameters measured by the separation component instrument and the oil line instrument and the water line instrument are used to determine the oil-in-water concentration. For example, an interface level parameter, a flow rate parameter of the oil line, and a flow rate parameter of the water line are used by the computational device to determine the volume of the oil and the volume of the water to in turn determine the oil-in-water concentration from the determined volumes over the certain period of time.

In another embodiment, the parameters measured by the separation component instrument and the oil line instrument and the inlet line instrument are used to determine the oil-in-water concentration. For example, an interface level parameter, a flow rate parameter of the oil line, and a flow rate parameter of the inlet line are used by the computational device to determine the volume of the oil and the volume of the treated water stream to in turn determine the oil-in-water concentration from the determined volumes over the certain period of time.

In another embodiment, the parameters measured by the separation component instrument and the water line instrument and the inlet line instrument are used to determine the oil-in-water concentration. For example, an interface level parameter, a flow rate parameter of the water line, and a flow rate parameter of the inlet line are used by the computational device to determine the volume of the water and the volume of the treated water stream to in turn determine the oil-in-water concentration from the determined volumes over the certain period of time.

The isolation valve 226 may be used to isolate some or all of the flow through the coalescer 222. This may be useful in some instances, e.g., to isolate the coalescer 222 from the treated water stream, to minimize clogging, fouling, or other maintenance and/or damage issues associated with use of the coalescer 222, to use the coalescer 222 only periodically, e.g., as a backup to another OIWM portion, to use as a reference for calibrating a sensor of another OIWM portion, to use as a reliability second check, etc. Isolating the coalescer 222 and related components may minimize system wear-and-tear. Those of skill in the relevant art will appreciate that other designs may be available from separation component equipment vendors. Further, redundant OIWM portions may be installed in parallel to improve the overall reliability of the OIWM system 200. All such alternate embodiments are considered within the scope of the present disclosure.

The inlet line 218 may pass at least a portion (second portion) of the treated water stream along a treated water line 242 to a second OIWM portion 221 of the OIWM system 200, the second OIWM portion 221 comprising an isolation valve 244 and an OIWM sensor 246 operatively coupled to the treated water line 242. The OIWM sensor 246 may be selected from a fluorescence sensor, an acoustic sensor, and an optical sensor. The treated water line 242 may be configured to receive the separated water portion from the coalescer 222 via the water line 238 after or downstream of the second OIWM portion 221 and may pass the resulting treated water stream out of the OIWM system 200, e.g., as reinjection water, as discharge water, as recirculation water, etc. Some embodiments may be configured to optionally select the destination of the resulting stream based on measurements calculated by the computational device (not shown).

It will be understood that OIWM system 200 shown in FIG. 2 has been simplified to assist in explaining various embodiments of the present techniques. Accordingly, in embodiments of the present techniques numerous devices not shown or specifically mentioned can further be implemented. Such devices can include additional flow meters. Flow meters as discussed herein may be selected from orifice flow meters, mass flow meters, ultrasonic flow meters, venturi flow meters, and combinations thereof. Additionally, the flow at each outlet from the coalescer 222 may be controlled by subsequent process equipment (e.g., using pumps through pump speed control, using inlet and/or outlet control valves, etc.) located elsewhere in the OIWM system 200. The schematic of FIG. 2 is not intended to indicate that the system is to include all of the components shown in FIG. 2. For example, some embodiments of the water treatment portion 202 may comprise single-stage deoiling hydrocyclones. Further, any number of additional components may be included within the OIWM system 200 depending on the details of the specific implementation. For example, one or more controllers may be added as described herein, the length of the coalescer 222 can be extended, e.g., by adding additional coalescers and/or coalescing components in series and/or parallel, to increase and improve oil/water separation. These and other modifications will be apparent to those of skill in the relevant art and are considered within the scope of the present disclosure.

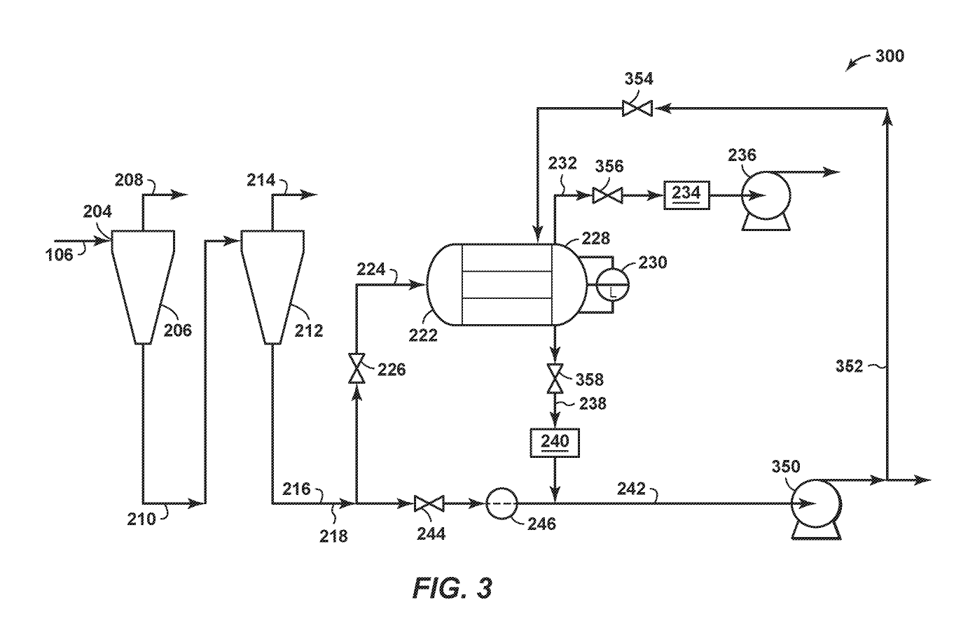

FIG. 3 is a schematic diagram of another embodiment of a system including an OIWM system 300. The components of FIG. 3 are substantially the same as the corresponding components of FIG. 2 except as otherwise noted. The OIWM system 300 includes a pump 350 disposed along the treated water line 242 to pass the resulting stream of treated water, including the second stream of the separated water portion from the first OIWM portion, out of the OIWM system 300, e.g., as reinjection water, as discharge water, as recirculation water, etc. The OIWM system 300 further comprises a back-flushing line 352 in fluid communication with the treated water line 242 and having a valve 354 operatively coupled thereto to pass at least a portion of the resulting stream of treated water in the treated water line 242 to the coalescer 222. In other embodiments, the back-flushing line 352 may be in fluid communication with water line 238 to pass at least a portion of the second stream of the separated water portion directly to the coalescer 222. In other embodiments, the back-flushing line 352 may be in fluid communication with the separation component instrument 230 and used to clean the instrument. The back-flushing line 352 may be optionally utilized, e.g., to remove clogging, fouling, sand, debris, and/or other undesirables from the coalescer 222. When permitted by the valve 354, the pump 350 may pass a back-flushing flow along the back-flushing line 352 to the coalescer 222. As would be understood by those of skill in the relevant art, embodiments utilizing a differing separation component, e.g., membranes, may be altered as needed to accommodate the back-flushing flow described above. Alternately or additionally, a back-flushing line (not shown) with a valve (not shown) operatively coupled thereto may pass at least a portion of the treated water stream from treated water line 242 to the OIWM sensor 246 to clean the OIWM sensor 246 to remove clogging, fouling, sand, debris and/or other undesirable materials that may interfere with the oil-in-water concentration determination.

FIG. 3 further includes control valves 356 and 358 on the oil line 232 and the water line 238, respectively. The control valves 356 and 358 may be configured to regulate the fluid velocity in the oil line 232 and the water line 238. The control valves 356 and 358 can indirectly control the oil/water portions in the plenum and/or chamber 228. The interface level, for example, between oil and water phases, can be detected in the plenum 228 at the separation component instrument 230. In response to a signal from the separation component instrument 230, e.g., indication that the oil-water interface has exceeded a predetermined threshold, a control signal may be generated by a controller (not shown) to throttle, open, or close one or more of the control valves 356 and 358 controlled with the same or different controllers (not shown). Other embodiments may use the isolation valve 226 in a similar manner, as would be understood by those of skill in the relevant art.

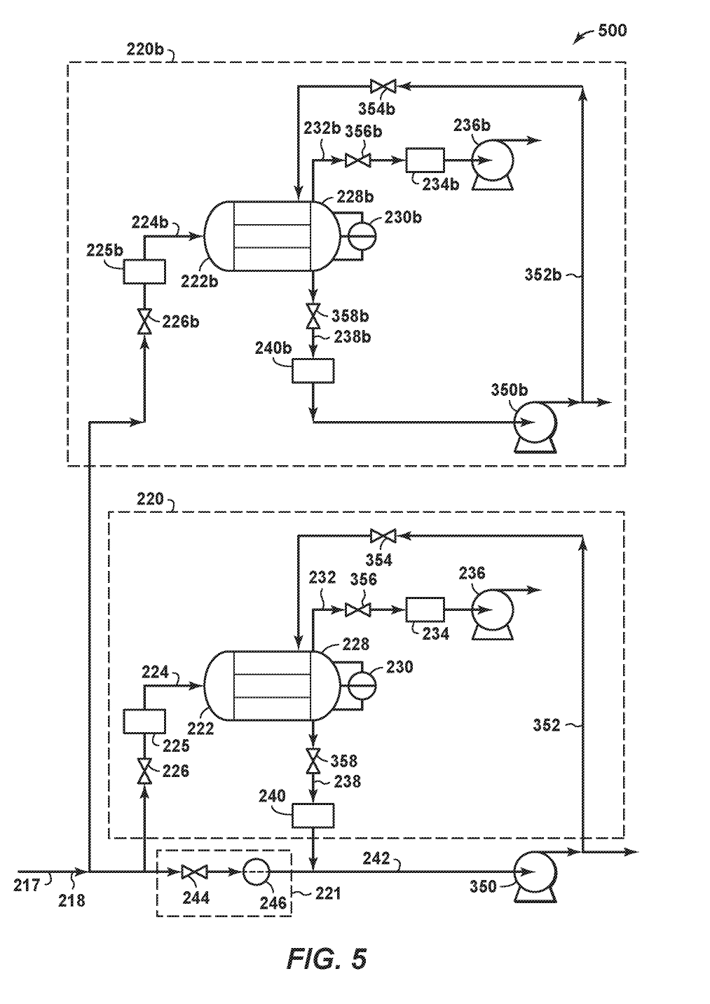

In some embodiments, the OIWM system may include a third OIWM portion 220b including an OIWM portion similar to the first OIWM portion 220 and arranged in parallel with the first OIWM portion 220 such that a third portion of the treated water stream enters the third OIWM portion 220b. FIG. 5 is a schematic diagram of OIWM system 500 depicting a third OIWM portion 220b in parallel with the first OIWM portion 220. The third OIWM portion 220b includes similar components as the first OIWM portion 220 and are denoted with a “b” designation, e.g., isolation valve 226b, inlet line 224b, coalescer 222b, plenum or chamber 228b, separation component instrument 230b, oil line 232b, water line 238b, instruments 234b, 240b, 225b, pumps 236b, 350b, isolation valves 244b, 354b, 356b, 358b, and back-flushing line 352b. The separated water portion and the separated oil portion of the third OIWM portion may be utilized in a similar way as with the first OIWM portion.

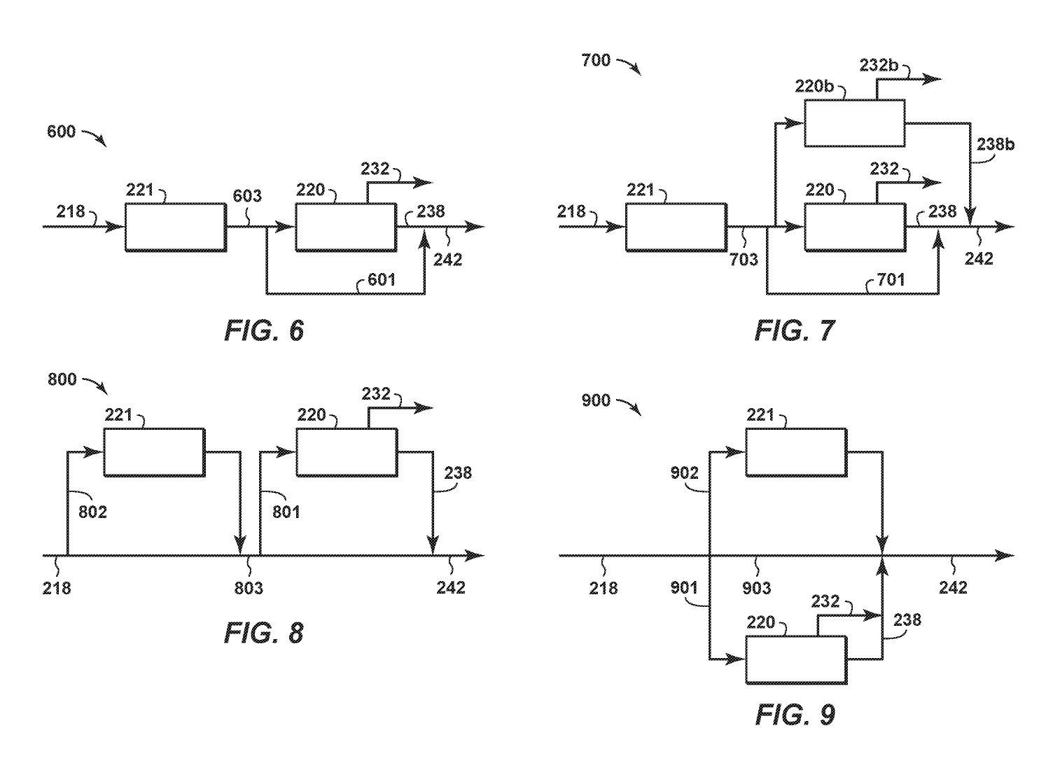

FIG. 6 is a block diagram of OIWM system 600 depicting two OIWM portions 220, 221 arranged in series with a bypass line 601 off of the main flow line 603 such that a portion of the treated water within the main flow line 603 may be introduced periodically to the first OIWM portion 220. The separated water line 238 may be combined with the flow via the bypass line 601 to form the treated water line 242.

FIG. 7 is a block diagram of OIWM system 700 depicting three OIWM portions 220, 220b, and 221. Second OIWM portion 221 is arranged in series with first OIWM portion 220 and a third OIWM portion 220b (similar to the third OIWM portion 220b depicted in more detail in FIG. 5) is arranged in parallel with the first OIWM portion 220. The OIWM system 700 includes a bypass line 701 off the main flow line 703 such that a portion of the treated water may be introduced periodically to the first OIWM portion 220 and/or the third OIWM portion 220b. The separated water lines 238, 238b may be combined with the flow via the bypass line 701 to form the treated water line 242. It is understood that the separated water portion and the separated oil portion of the third OIWM portion may be utilized in a similar way as with the first OIWM portion. Providing the third OIWM portion 220b in parallel with the first OIWM portion 220, provides redundancy for the first OIWM portion 220.

FIG. 8 is a block diagram of OIWM system 800 depicting two OIWM portions 220, 221. As depicted, both the first OIWM portion 220 and the second OIWM portion 221 are positioned within side stream lines 801, 802, respectively. Side stream line 802 is positioned off the main flow line 803 prior to side stream line 801.

FIG. 9 block diagram of OIWM system 900 depicting two OIWM portions 220, 221. As depicted, both the first OIWM portion 220 and the second OIWM portion 221 are positioned within side stream lines 901, 902, respectively. Side stream lines 901, 902 are positioned off the main flow line 903 at the same location but on opposite sides of the main flow line 903.

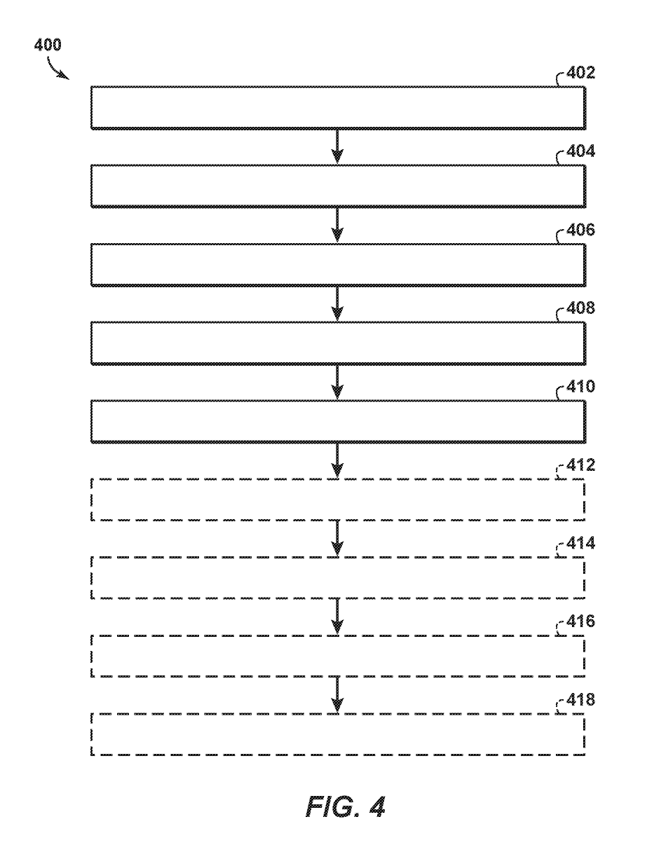

FIG. 4 is a block diagram showing a method 400 of monitoring an oil-in-water concentration of treated water in a subsea processing system. The method 400 begins at block 402 with passing at least a portion (first portion) of treated water into the first OIWM portion, the treated water may be obtained by passing produced water through a water treatment portion e.g., treated water stream passes via outlet line 216 from the water treatment portion 202 to the inlet line 224 of the first OIWM portion 220 via main inlet line 218 of FIG. 2.

At block 404, the first OIWM portion separates the treated water stream (first portion) into a separated oil portion and a separated water portion. As described above, separation may be obtained by using a separation component which may use any of a variety of techniques known in the art, including one or more coalescers, membrane-based filters, etc. Once separated, the separated oil portion and the separated water portion may be temporarily retained in a plenum or chamber, e.g., the plenum 228 of FIG. 2.

At block 406, a parameter (e.g., an interface level) of the separation component plenum is measured and at least two additional parameters associated with the separated oil portion, the separated water portion, and the portion (first portion) of the treated water stream passed to the first OIWM portion are measured. The parameters are used to determine volumes which are in turn used to determine the oil-in-water concentration as discussed further herein.

At block 408, a result (first result) is produced indicating an oil-in-water concentration of the portion (first portion) of the treated water stream using the parameters measured at block 406

At block 410, the result (first result) indicating the oil-in-water concentration of the portion (first portion) of the treated water stream passed to the first OIWM portion is compared to a result (second result) indicating the oil-in-water concentration of a portion (second portion) of the treated water stream passed to a second OIWM portion including a primary OIWM sensor. The oil-in-water concentrations determined for the treated water may be used for determining the accuracy or abnormal operation of the second OIWM portion, abnormal operation of the first OIWM portion, calibrating the OIWM sensor of the second OIWM portion, determining an efficiency of the water treatment portion, and/or another metric indicating the efficacy of the overall system, e.g., the OIWM system 200 of FIG. 2. The comparison may alternately or additionally indicate the need for repair, maintenance, calibration, and/or replacement of a portion of the OIWM system. One or more of these parameters may also trigger a response action, e.g., throttling, opening, or closing one or more valves within the OIWM system. For example, a higher oil-in-water concentrations may be permitted for reinjection operations rather than for discharge operations. Consequently, exceeding a predetermined oil-in-water concentration level may trigger one or more additional actions, such as stopping production, redirecting the treated water stream, generating an alarm, etc. These and other examples will be readily apparent to those of skill in the relevant art and are considered within the scope of the present disclosure

The dashed lines at block 412 indicate an optional portion of the method which may include using at least a portion of the separated water portion from the first OIWM portion as at least a portion of a reinjection water stream, a discharge water stream, or both.

The dashed lines at block 414 indicate an optional portion of the method which may include passing at least a portion of the separated water portion from the first OIWM portion to the separation component and/or the second OIWM sensor as a back-flushing stream and back-flushing the separation component and/or the second OIWM sensor with the back-flushing stream. This may be helpful to remove any clogging, fouling, etc. that may occur during the lifecycle of the separation component, and/or clean the second OIWM sensor head to ensure the sensor performs properly. The back-flushing stream may be stopped upon completion of back-flushing operations by closing an isolation valve.

The dashed line at block 416 indicate an optional portion of the method which may include receiving another portion (third portion) of the treated water as an inlet stream for a third OIWM portion used to separate oil from the inlet stream to the third OIWM portion providing redundancy to the first OIWM portion. The third OIWM portion may be used to determine oil-in-water concentrations of treated water in addition to or alternatively to the first OIWM portion.

The dashed line at block 418 indicates an optional portion of the method which may include calibrating the OIWM sensor of the second OIWM portion using the comparison of the results indicating the oil-in-water concentrations of the first OIWM portion and the second OIWM portion and then stopping the portion of the treated water stream from passing to the first OIWM portion by closing the isolation valve operatively coupled to the inlet line to the separation component.

The process flow diagram of FIG. 4 is not intended to indicate that the steps of the method 400 are to be executed in any particular order, or that all of the steps of the 400 are to be included in every case. Further, any number of additional steps not shown in FIG. 4 may be included within the method 400, depending on the details of the specific implementation.

The methods may also include passing at least a portion of the separated oil portion to a produced oil stream. With respect to the OIWM systems depicted in FIGS. 5 and 7, the method may include passing a third portion of the treated water to a third OIWM portion and separating the third portion of the treated water into a separated oil portion and a separated water portion using the separation component within the third OIWM portion. The third OIWM portion may be used to measure an interface level in the separation component of the third OIWM portion and at least two additional parameters associated with the separated oil portion, the separated water portion, and the third portion of the treated water. A third result indicating an oil-in-water concentration of the third portion of the treated water may be produced using the interface level and the at least two additional parameters of the third OIWM portion. The third result indicating the oil-in-water concentration of the third portion of the treated water may be compared to the second result indicating the oil-in-water concentration of the second portion of the treated water. The first OIWM portion and the third OIWM portion may be operated simultaneously or sequentially by turning on/off associated isolation valves.

While the present techniques may be susceptible to various modifications and alternative forms, the embodiments discussed above have been shown only by way of example. However, it should again be understood that the techniques is not intended to be limited to the particular embodiments disclosed herein. Indeed, the present techniques include all alternatives, modifications, and equivalents falling within the true spirit and scope of the appended claims.

What is claimed is:

1. An oil-in-water monitoring (OIWM) system, comprising:
an inlet line configured to pass a treated water stream into a first OIWM portion, wherein the treated water stream comprises oil and water;
a separation component in fluid communication with the inlet line and configured to separate the treated water stream into a separated oil portion and a separated water portion, wherein the separation component comprises a plenum including the separated oil portion and the separated water portion;
a separation component instrument operatively coupled to the plenum and configured to measure a parameter associated with the separated oil portion, the separated water portion, or both within the plenum;
an oil line in fluid communication with the separation component and configured to pass a first stream comprising the separated oil portion out of the first OIWM portion;
a water line in fluid communication with the separation component and configured to pass a second stream comprising the separated water portion out of the first OIWM portion;
at least two of: an oil line instrument operatively coupled to the oil line and configured to measure a parameter associated with the separated oil portion within the oil line, a water line instrument operatively coupled to the water line and configured to measure a parameter associated with the separated water portion within the water line, and an inlet line instrument operatively coupled to the inlet line and configured to measure a parameter associated with the treated water stream within the inlet line; and
a computational device operatively coupled to the separation component instrument and at least two of: the oil line instrument, the water line instrument, and the inlet line instrument, the computational device configured to output an oil-in-water concentration of the treated water stream using the parameters measured by the separation component instrument and at least two of the other instruments.

2. The system of claim 1, wherein the separation component instrument is a level meter and the parameter measured is an interface level in the plenum, the oil line instrument is a first flow meter and the parameter measured is the flow rate of the separated oil portion within the oil line, the water line instrument is a second flow meter and the parameter measured is the flow rate of the separated water portion within the water line, and the oil-in-water concentration of the treated water stream is determined using the interface level, the flow rate of the separated oil portion within the oil line and the flow rate of the separated water portion within the water line, and wherein the first flow meter, the second flow meter, and the level meter are configured to transmit a representative signal to the computational device.

3. The system of claim 1, wherein the separation component instrument is a level meter and the parameter measured is an interface level in the plenum, the oil line instrument is a first flow meter and the parameter measured is the flow rate of the separated oil portion within the oil line, the inlet line instrument is a third flow meter and the parameter measured is the flow rate of the treated water stream within the inlet line, and the oil-in-water concentration of the treated water stream is determined using the interface level, the flow rate of the separated oil portion within the oil line and the flow rate of the treated water stream within the inlet line, and wherein the first flow meter, the third flow meter, and the level meter are configured to transmit a representative signal to the computational device.

4. The system of claim 1, wherein the separation component instrument is a level meter and the parameter measured is an interface level in the plenum, the water line instrument is a second flow meter and the parameter measured is the flow rate of the separated water portion within the water line, the inlet line instrument is a third flow meter and the parameter measured is the flow rate of the treated water stream within the inlet line, and the oil-in-water concentration of the treated water stream is determined using the interface level, the flow rate of the separated water portion within the water line and the flow rate of the treated water stream within the inlet line, and wherein the second flow meter, the third flow meter, and the level meter are configured to transmit a representative signal to the computational device.

5. The system of claim 1, wherein the computational device is further configured to output an average of determined oil-in-water concentrations for the treated water stream over a given time.

6. The system of claim 1, wherein the oil line is configured to pass the first stream to a produced oil stream.

7. The system of claim 1, wherein the water line is configured to pass at least a portion of the second stream into the separation component as a back flushing flow.

8. The system of claim 1, wherein the oil-in-water concentration of the first OIWM portion is used to calibrate a second OIWM portion comprising an OIWM sensor, the first OIWM portion configured to be isolatably coupled in parallel to the second OIWM portion.

9. The system of claim 1, wherein the separation component is a coalescer, membrane-based filter, or both.

10. A method of monitoring an oil-in-water concentration of treated water in a subsea processing system, comprising:
passing a first portion of the treated water to a first oil-in-water monitoring (OIWM) portion;
separating the first portion of the treated water into a separated oil portion and a separated water portion using a separation component within the first OIWM portion;
measuring an interface level in the separation component and at least two additional parameters associated with the separated oil portion, the separated water portion, and the first portion of the treated water;
producing a first result indicating an oil-in-water concentration of the first portion of the treated water using the interface level and the at least two additional parameters; and
comparing the first result to a second result indicating an oil-in-water concentration of a second portion of the treated water, wherein the second result is obtained at a second OIWM portion including an OIWM sensor.

11. The method of claim 10, further comprising:
using at least a portion of the separated water portion as at least a portion of a reinjection water stream, a discharge water stream, or both.

12. The method of claim 10, further comprising:
passing at least a portion of the separated water portion to the separation component as a back-flushing stream; and
back-flushing the separation component with the back-flushing stream.

13. The method of claim 10, further comprising passing a third portion of the treated water to a third oil-in-water monitoring (OIWM) portion including:
a separation component in fluid communication with an inlet line and configured to separate the third portion of the treated water into a separated oil portion and a separated water portion, wherein the separation component comprises a plenum including the separated oil portion and the separated water portion;
a separation component instrument operatively coupled to the plenum and configured to measure a parameter associated with the separated oil portion, the separated water portion, or both within the plenum;
an oil line in fluid communication with the separation component and configured to pass a third stream comprising the separated oil portion out of the third OIWM portion;
a water line in fluid communication with the separation component and configured to pass a fourth stream comprising the separated water portion out of the third OIWM portion; and
at least two of: an oil line instrument operatively coupled to the oil line of the third OIWM portion and configured to measure a parameter associated with the separated oil portion within the oil line, a water line instrument operatively coupled to the water line of the third OIWM portion and configured to measure a parameter associated with the separated water portion within the water line, and an inlet line instrument operatively coupled to the inlet line to the third OIWM portion and configured to measure a parameter associated with the treated water stream within the inlet line; and
separating the third portion of the treated water into the separated oil portion and the separated water portion using the separation component within the third OIWM portion;
measuring an interface level in the separation component of the third OIWM portion and at least two additional parameters associated with the separated oil portion, the separated water portion, and the third portion of the treated water of the third OIWM portion;
producing a third result indicating an oil-in-water concentration of the third portion of the treated water using the interface level and the at least two additional parameters of the third OIWM portion; and
comparing the third result to the second result indicating the oil-in-water concentration of the second portion of the treated water.

14. The method of claim 10, further comprising:
passing at least a portion of the separated oil portion to a produced oil line comprising a produced oil stream.

15. The method of claim 10, further comprising:
calibrating the OIWM sensor of the second OIWM portion using the comparison; and
stopping the treated water from passing to the first OIWM portion.

16. An oil-in-water monitoring (OIWM) system, comprising:
a first OIWM portion configured to receive at least a first portion of a treated water stream passing from an outlet of a water treatment portion, wherein the first OIWM portion is further configured to measure a plurality of parameters used to determine an oil-in-water concentration of the treated water stream, the first OIWM portion comprising:
a coalescer configured to receive the first portion of the treated water stream via an inlet line, wherein the coalescer is configured to separate the first portion of the treated water stream into a separated oil portion and a separated water portion, and wherein the coalescer comprises a plenum including the separated oil portion and the separated water portion;
a coalescer instrument operatively coupled to the plenum and configured to measure an interface level within the plenum;
an oil line in fluid communication with the coalescer and configured to pass a first stream comprising the separated oil portion out of the first OIWM portion;
a water line in fluid communication with the coalescer and configured to pass a second stream comprising the separated water portion out of the first OIWM portion;
at least two of:
a first flow meter operatively coupled to the oil line and configured to measure a first flow rate associated with the separated oil portion within the oil line,
a second flow meter operatively coupled to the water line and configured to measure a second flow rate associated with the separated water portion within the water line, and
a third flow meter operatively coupled to the inlet line and configured to measure a third flow rate associated with the first portion of the treated water stream within the inlet line; and
a computational device operatively coupled to the coalescer instrument and at least two of: the first flow meter, the second flow meter, and the third flow meter, the computational device configured to calculate an oil-in-water concentration of the first portion of the treated water stream using the interface level and at least two of: the first flow rate, the second flow rate, and the third flow rate; and
a second OIWM portion configured to receive at least a second portion of the treated water stream passing from the outlet of the treated water portion, wherein the second OIWM portion includes an OIWM sensor and is further configured to determine an oil-in-water concentration of the second portion of the treated water stream.

17. The system of claim 16, further comprising:
an isolation valve operatively coupled to the inlet line to the coalescer of the first OIWM portion for stopping the first portion of the treated water stream from passing into the coalescer.

18. The system of claim 16, wherein the first OIWM portion and the second OIWM portion are arranged in parallel.

19. The system of claim 16, wherein the coalescer comprises a back-flushing inlet configured to clean at least a portion of the coalescer, the coalescer instrument, or both with a received back-flushing stream.

20. The system of claim 16, wherein the computational device is further configured to compare an average of at least a portion of the determined oil-in-water concentrations for the first portion of the treated water stream with an average of at least a portion of the determined oil-in-water concentrations for the second portion of the treated water stream.

21. The system of claim 16, wherein the first OIWM portion is configured to pass at least a portion of the second stream to a reinjection water line.
